(19) United States
(12) Patent Application Publication
(10) Pub. No.: US 2002/0086430 A1
(43) Pub. Date: Jul. 4, 2002

(54) Detection technology in agriculture operations

(76) Inventor: Michael J. Hopmeier, Mary Ester, FL (US)

Correspondence Address: HELLER EHRMAN WHITE & MCAULIFFE LLP, SUITE 300, 101 ORCHARD RIDGE DR., GAITHERSBURG, MD, 20878-1917, US

(21) Appl. No.: 10/029,023

(22) Filed: Dec. 28, 2001

Related U.S. Application Data
(60) Provisional application No. 60/258,309, filed on Dec. 28, 2000.

(51) Int. Cl. G01N 33/02

(52) U.S. Cl. 436/20; 422/68.1; 435/5

(57) ABSTRACT

Billions of dollars are being spent in the development and deployment of technologies for monitoring environmental biological and chemical contaminants to support early detection and reaction to potential biological warfare and terrorism. To date, little commercial value has been seen in these technologies. However, direct application of these technologies for monitoring, in real time, the growth environment found in high density farming applications (i.e. poultry, pigs, veal, etc.) offers the potential for considerable improvement in yields, reduced use of antibiotics and other prophylactic pharmaceuticals and reduced potential for outbreaks of drug resistant disease.

### REFERENCE TO RELATED APPLICATIONS

This application claims priority to U.S. Provisional application, serial No. 60/258,309, entitled Biological Agent Detection Technology in High Density Agriculture Operations, filed Dec. 28, 2000.

### BACKGROUND

1. Field of the Invention

The invention relates to apparatus and methods for monitoring environmental biological and chemical contaminants in a defined area. In particular, the invention relates to such apparatus and methods for use in agricultural operations.

2. Description of the Background

In high density farming animals live almost on top of each other in minimal space with integrated and coordinated support systems such as waste disposal, air circulation and feeding. A vast array of pharmaceuticals, mostly antibiotics, are used to reduce the prevalence and severity of disease, and concomitantly increase yield. However, this practice has of late been coming under fire for a variety of reasons. The use of prophylactic pharmaceuticals (PP) has multiple problems.

The use of PP has been a contributing factor to the development and emergence of drug resistant pathogens. The environment in which they are used is almost perfect for forced evolution of pathogens. There is concern that these PP remain in the food, even after processing, and are therefore entering human systems with unknown end results. The added costs of procurement and dissemination of these PP is high, and adds to the bottom line costs of production. This cost is not merely the that of acquisition but also maintenance of records, compliance with regulatory and oversight bodies and logistics, transport and processing of wastes.

The PP used are broad spectrum and seldom targeted to a particular disease and, consequently may not be optimized for the particular situation in which they are utilized. Consequently, costs and efficacy are sub optimized. Of principal concern, however, is public perception of use of broad band antibiotics as a growth media. This perception has already resulted in the banning of this practice in Australia and several parts of Europe, and is currently under consideration in the US. Should this occur, cost of production, and hence end product will almost certainly increase and lead to increased consumer costs. This will result from decreased yields caused by smaller animals, a greater incidence of disease, smaller commercial yields per animal or crop, increased waste, etc.

### SUMMARY OF THE INVENTION

The present invention overcomes the problems and disadvantages associated with current strategies and designs and provides effective methods and systems for monitoring for the presence of chemical or biological agents in agricultural operations. More specifically, the present invention relates to methods and systems to monitor and prevent or at least minimize harm to the agricultural operation.

One embodiment of the invention is directed to methods of monitoring agricultural operations in defined geographical areas, which comprises dispersing a plurality of sensors within the geographical area wherein each sensor can detect a chemical or biological agent that may pose a threat to the operation; and monitoring the plurality of sensors for the presence of one or more of the chemical or biological agents. Appropriate sensors are commercially available or can be designed to meet the needs of the specific agricultural operation.

Another embodiment of the invention is directed to methods of monitoring agricultural operations in a geographical area comprising dispersing a plurality of sensors within the area wherein each sensor can detect a chemical or biological agent that may pose a threat to the agricultural operation; monitoring the plurality of sensors for the presence of one or more of the chemical or biological agents; and taking action to protect the agricultural operation upon detection of a chemical or biological agent by one or more of the sensors.

Another embodiment of the invention is directed to systems designed to detect potentially harmful chemical or biological agents that enter agricultural operations. Such systems comprise a plurality of sensors designed to detect a chemical or biological agent, wherein the sensors are dispersed throughout the agricultural operation; and a control station connected to each sensor which provides information to a user on the activity of the plurality of sensors. The invention may further comprise a means to segregate a portion of the agricultural operation from the entirety of the operation. Preferably the means is operably connected to the control station and the control station is a computer.

Other embodiments and advantages of the invention are set forth, in part, in the following description including the figures, and, also in part, will be obvious from this description, or may be learned from the practice of the invention.

### DESCRIPTION OF THE INVENTION

As embodied and broadly described herein, the present invention is directed to effective methods and systems for monitoring for the presence of chemical or biological agents in agricultural operations. More specifically, the present invention relates to methods and systems to monitor and prevent or at least minimize harm to the agricultural operation.

Considerable resources are being spent to develop environmental sensors and systems to support the early detection and reaction against potential biological warfare agents and terrorism threats. It has been surprisingly discovered that similar sensors and systems can be modified to have a non-military function, specifically, to monitor crops and animals in agricultural operations for harmful chemical and/or biological agents. The ability to respond immediately to the site of a potential outbreak and isolate or eradicate the outbreak, can wipe out a problem before it becomes massive and uncontrollable. As measures already exist to wipe out the infection, what remains is the ability to provide early detection that is as accurate as it is prompt.

Conventional sensing and identifying involves the detection of unknown agents nearly in uncontrolled areas, such as open air around cities and in buildings, for a wide array of chemicals and pathogens. These sensors must take into account the hugely varying backgrounds associated with changes in seasons as well as various outbreaks of relatively innocuous diseases (e.g. influenza, rhinovirus), while still being able to identify emerging or anomalous disease signatures. This technology can be applied to agricultural situations such as high-density farming for a similar service, to provide early warning of threats to crops or animal populations. In such operations, typically there is a fixed population under tight control and scrutiny with very little geographic displacement or movement. There also exists a tightly controlled food, air, water, waste and effluent streams, with, in normal operations, a limited number of pathogens of interest, or at least an ability to set sensors to maximize detection for those pathogens that are of greatest concern.

Accordingly, one embodiment of the invention is directed to methods of monitoring an agricultural operation in a geographical area comprising dispersing a plurality of sensors within the geographical area wherein each sensor can detect a chemical or biological agent that may pose a threat to the agricultural operation; and monitoring the plurality of sensors for the presence of one or more of the chemical or biological agents.

Chemical agents that can be detected include solids and fluids such as gasses, vapors or liquids. Specific agents that can be detected, include, but are not limited to, benzene, chlorine, chloroform, fluorine, harmful aromatics and hydrocarbons, metals such as lead and sodium, methane, ozone, any of a variety of petroleum-based materials, propane, sulfur dioxide and any of a variety of industrial or other pollutants, and weaponized substances such as serin gas. Biological agents that can be detected include, for example, whole or parts of various pathogenic or otherwise harmful microorganisms such as bacteria (e.g. gram negative and gram positive, aerobic and anaerobic), fungi, parasites (e.g. Ameba, Leishmania, Nematodes, Protozoa), spores (e.g. anthrax), and viruses (e.g. pox virus, hepatitis, rabies, rhinovirus), or whole or parts of multi-celled organisms such as beetles, fleas, fungus, insects, mites, ticks and other pests. Specific examples include, but are not limited to species of Escherichia, Bacillis, Pseudomonas, Staphylococcus, Streptococcus, and Vibrio. Disease organisms that can be detected include, but are not limited to, botulism, cholera, diphtheria, dysentery, and salmonella.

Many sensors which may be used are commercially available or can be designed to the specific agricultural operation. For example, appropriate sensors may detect changes in pH, temperature, RedOx potential, combustibility, ozone concentration, ion or free radical content, the presence of absence of radiation including, but not limited to, visible light, bioluminescence, fluorescence, infrared, ultraviolet and radio waves, changes in absorbency, transmission, or scattering, and the presence of absence of specific chemical or biological materials. Biological materials that can be detected include infections and infectious agents, enzyme activities and levels, characteristic waste materials or substances, gaseous substances, and combinations thereof. Sensors that can be used in the methods and systems of the invention are generally commercially available and many are described in U.S. Pat. Nos. 4,596,697; 4,824,206; 4,892,383; 4,935,207; 4,948,722; 5,004,914; 5,028,395; 5,047,213; 5,055,268; 5,078,855; 5,109,442; 5,10,393; 5,205,292; 5,284,146; 5,294,402; 5,310,526; 5,362,975; 5,393,401; 5,418,058; 5,443,354; 5,496,522; 5,569,838, 5,593,854; 5,597,534; 5,618,493; 5,686,300; 5,719,033; 5,741,634; 5,796,097; 5,822,473; 5,827,748; 5,880,352; 5,910,286; 5,958,340; 5,972,638; 6,035,705; 6,201,980; 6,269,703; and 6,303,386. However, sensors can also be specifically designed to detect one or a plurality of chemical and/or biological agents as most useful for the particular agricultural operation. Sensors can provide simple detection information, concentration information and also trend information in situations where absolute amounts of biological or chemical substances and not as important as changes and rate of changes.

The agricultural operation may comprise raising plants or animals, or any other operation (e.g. high-density agricultural operation), conducted in a predetermined geographical area (e.g. caves, farms, fenced in areas, fields, vineyards, or any confined or partially confined areas). Plants that are raised on an agricultural operation include, but are not limited to, fruits, fungi, grains, soybeans, trees, vegetables, and combinations thereof. Preferred plants include corn, mushrooms, rice, soybeans, and wheat. Preferred animals include cattle, chickens, ducks, horses, pigs, and sheep. Also preferably, sensors are placed at the points where materials enter or exit the agricultural operation. Those materials include, but are not limited to, air, bedding materials, biological waste materials, effluent, feed, fertilizer, soil, and water, and even within the animals or crops that make up the operation. Agricultural areas include, but are not limited to, any defined geographical area, such as a pen, a corral, a yard, a fenced-in area, a barn, an acreage, or area of planted crops. The sensors, which comprise as many as needed for the particular geographical area and operational design, may comprise as few as three, at least five, at least twenty five, at least fifty, or more as needed. Many types of chemical sensors are commercially available that are designed to detect agents such as, but not limited to, carcinogens, contaminants, poisons, pollutants, toxins, and combinations thereof. Biological sensors are also commercially available that can detect bacteria, fungi, parasites, viruses, and combinations thereof.

The invention is also directed to the placement of said sensors in breeding, growing, and raising crops and animals and also in monitoring the populations. Preferably, sensors are placed at locations where water and food or fertilizer enter the geographic area, up-wind of the prevailing winds for air-borne detection, where waste such as liquid run off and solids such as bio-waste or manure, leave the area, and bedding or other common areas. With constant, nearly constant or periodic monitoring, when an indication of a disease appears, immediate, often optimal, therapy can be provided. As treatment is provided when most useful, the control and eradication of the harm or disease is mostly assured. Periods may be seconds, minutes, hours, days, moths or even longer. Determination on length of the period lies with the requirement for action upon detection of harmful agents. Sensors may periodically or constantly monitor these controlled environments and provide information, not only on outbreak but potentially on the overall health of the population, or that portion of the population, thereby allowing for real-time, closed-loop control of the production process.

Another embodiment of the invention is directed to methods of monitoring an agricultural operation in a geographical area comprising dispersing a plurality of sensors within the area wherein each sensor can detect a chemical or biological agent that may pose a threat to the agricultural operation; monitoring the plurality of sensors for the presence of one or more of the chemical or biological agents; and taking action to protect the agricultural operation upon detection of a chemical or biological agent by one or more of the plurality of sensors. The action taken is designed to protect the operation from harm or at least limit that harm from the rest of the operation and from nearby operations. When a sensor detects a potentially harmful agent, the action that may be taken includes, but is not limited to, treating the animals or plants of the agricultural operation with an agent that inactivates the chemical or biological agent detected; treating all or a portion of the agricultural operation with a prophylactic that prevents harm from the chemical or biological agent detected; destroying all or selected portions of the agricultural operation; or any combination thereof.

Another embodiment of the invention is directed to systems designed to detect potentially harmful chemical or biological agents that enter an agricultural operation. Such systems comprise a plurality of sensors designed to detect a chemical or biological agent, wherein the plurality is dispersed throughout the agricultural operation; and a control station connected to each sensor which provides information to a user on the activity of the plurality of sensors. The control station may be a computer that monitors the sensors. When a sensor detects a potentially harmful chemical or biological agent, an alarm can be sounded to alert the operator. The operator can take immediate action to protect the non-exposed portions of the operation and treat the exposed portion as necessary.

The invention may further comprise a means to segregate a portion of the agricultural operation from the entirety of the operation. Preferably the means is operably connected to the control station and can be operated by the user. An aspect of this embodiment is that control over all operations associated with detection of a potentially harmful agent can be centralized to one location and even one person. This maximizes efficiency and control over the operation and allows one individual or group to control multiple operations. Thus, another embodiment of the invention is a business model whereby monitoring and remedial action necessary to protect unexposed operations and treat exposed operations can be directed from a central facility by experienced professionals.

Additional embodiments of the invention include coupling sensor nets of the invention to other detection systems such as weather stations that monitor wind speed or direction and general weather patterns. In this way, contaminants that enter the agricultural operation can be tracked and their original location discovered. Further, multiple systems of the invention can be incorporated into broader systems that are larger in scope and encompass large geographical areas such as counties, regions, states and countries. Such systems of the invention can monitor and rapidly detect and identify biological and/or chemical agents that pose significant concern to more than one agricultural operation.

The following examples are offered to illustrate embodiments of the invention, but are not to be viewed as limiting the scope of the invention.

### EXAMPLES

### General System Designs of the Invention

One system of the invention is designed to monitor the health of a herd of dairy cattle. Sensors to detect agents of biological warfare such as cowpox are placed in a plurality of locations such as in the cattle's feed, water supply, and waste removal area, and in the general area to which the cattle are confined. Sensors can be designed to detect surface proteins of the virus, infected cells, or certain metabolic products. Upon detection of cowpox, the computer monitor notifies the user and the user immediately takes action. That action may be to segregate infected from non-infected cattle or to immediately begin treatment and/or prophylaxis (i.e. vaccination) of the herd.

Another system of the invention is designed to detect nerve toxins deliberately or accidentally administered to water supplies containing fish. Sensors are placed in a plurality of locations including the water intake areas, water exit areas, the bodies of the fish, in the soil at the bottom of the water, and in the water in general. Upon detection of the specific nerve toxin, the water supply and/or the fish are immediately treated with chemicals to neutralize or destroy the toxin.

Another system of the invention is designed for detection of ozone concentrations harmful to wheat. Ozone sensors are placed at key locations up wind of the crops. Upon detection of increased ozone levels, a gas is immediately releases into the area of the crops that destroy or otherwise complex the ozone preventing harm to the wheat. An alternative system can also be designed for the detection of harmful levels of radiation. Upon detection, shields are placed over the crop to prevent harmful exposure.

Another system of the invention can be designed to detect anthrax in the environment of a heard of sheep. Sensors that detect pathogenic anthrax spores are placed in a plurality of locations such as in the soil, water, food and air supply provided to the herd. Upon detection of anthrax spores, the herd can be sequestered to another location and treated before an infection can take hold that would require the animals to be destroyed.

Other embodiments and uses of the invention will be apparent to those skilled in the art from consideration of the specification and practice of the invention disclosed herein. All references cited herein for any reason, including all U.S. and foreign patents and patent applications, are specifically and entirely incorporated by reference. It is intended that the specification and examples be considered exemplary only, with the true scope and spirit of the invention indicated by the following claims.

1. A method of monitoring an agricultural operation in a geographical area comprising:
dispersing a plurality of sensors within said geographical area wherein each said sensor can detect a chemical or biological agent that may pose a threat to the agricultural operation; and
monitoring said plurality of sensors for the presence of one or more of said chemical or biological agents.

2. The method of claim 1 wherein the agricultural operation comprises raising plants or animals.

3. The method of claim 2 wherein the plants are selected from the group consisting of fruit crops, fungi, grain crops, trees, vegetable crops, and combinations thereof.

4. The method of claim 3 wherein the plants are selected from the group consisting of corn, mushrooms, rice, soybeans, wheat, and combinations thereof.

5. The method of claim 2 wherein the animals are selected from the group consisting of cattle, chickens, ducks, horses, pigs, sheep, and combinations thereof.

6. The method of claim 1 wherein the plurality of sensors are dispersed at the points where materials enter or exit the agricultural operation.

7. The method of claim 1 wherein the geographical area is selected from the group consisting of an acreage, a barn, a corral, a fenced-in area, a pen, a area of planted crops, a vineyard, a yard, and combinations thereof.

8. The method of claim 1 wherein the plurality of sensors comprises at least five sensors, at least twenty five sensors, or at least fifty sensors.

9. The method of claim 1 wherein the chemical agent is selected from the group consisting of carcinogens, contaminants, poisons, pollutants, toxins, and combinations thereof.

10. The method of claim I wherein the biological agent is selected from the group consisting of bacteria, fungi, parasites, viruses, and combinations thereof.

11. The method of claim 1 wherein monitoring comprises testing samples that enter into the agricultural operation.

12. The method of claim 11 wherein the samples are selected from the group consisting of samples of air, bedding materials, feed, fertilizer, soil, water, and combinations thereof.

13. The method of claim 1 wherein monitoring comprises testing samples that leave the agricultural operation.

14. The method of claim 13 wherein the samples are selected from the group consisting of samples of air, bedding materials, biological waste materials, effluent, soil, water, and combinations thereof.

15. A method of monitoring an agricultural operation in a geographical area comprising:
dispersing a plurality of sensors within said area wherein each said sensor can detect a chemical or biological agent that may pose a threat to the agricultural operation;
monitoring said plurality of sensors for the presence of one or more of said chemical or biological agents; and
taking action to protect the agricultural operation upon detection of a chemical or biological agent by one or more of said plurality of sensors.

16. The method of claim 15 wherein the action is selected from the group consisting of:
treating the animals or plants of the agricultural operation with an agent that inactivates the chemical or biological agent detected;
treating all or a portion of the agricultural operation with a prophylactic that prevents harm from the chemical or biological agent detected;
destroying all or selected portions of the agricultural operation; and
combinations thereof.

17. A system designed to detect potentially harmful chemical or biological agents that enter an agricultural operation comprising
a plurality of sensors designed to detect a chemical or biological agent, wherein said plurality is dispersed throughout the agricultural operation; and
a control station connected to each sensor which provides information to a user on the activity of said plurality of sensors.

18. The system of claim 17 wherein the control station is a computer.

19. The system of claim 17 further comprising a means to segregate a portion of the agricultural operation from the entirety of the operation.

20. The system of claim 19 wherein said means is operably connected to said control station.
